(19) United States
(12) Patent Application Publication
(10) Pub. No.: US 2023/0096856 A1
(43) Pub. Date: Mar. 30, 2023

(54) COMBINED TREATMENT REGIMEN OF ANESTHESIA/SEDATION WITH ADMINISTRATION OF PSYCHEDELIC DRUGS ASSOCIATED WITH REDUCTION IN NEUROPSYCHIATRIC ILLNESS

(71) Applicant: Pillar Clinical Research, LLC, Richardson, TX (US)

(72) Inventors: Brian Craig, Celeste, TX (US); Curtis Ponton, Santa Fe, NM (US)

(73) Assignee: Pillar Clinical Research, LLC, Richardson, TX (US)

(21) Appl. No.: 17/935,839

(22) Filed: Sep. 27, 2022

Related U.S. Application Data
(60) Provisional application No. 63/261,711, filed on Sep. 27, 2021.
(60) Provisional application No. 63/262,800, filed on Oct. 20, 2021.
(60) Provisional application No. 63/266,756, filed on Jan. 13, 2022.

(51) Int. Cl. A61K 31/4045; A61K 31/05; A61K 9/00; A61P 25/00

(52) U.S. Cl. CPC A61K 31/4045; A61K 31/05; A61K 9/0019; A61P 25/00

(57) ABSTRACT

A preferred embodiment is provided that utilizes psychedelics that induce the shortest possible hallucinogenic experience, to facilitate the use of fast-acting, short duration anesthetics that creates a state of unconsciousness that eliminates and/or minimizes conscious awareness and/or recall of the hallucinogenic experience. The preferred embodiment facilitates critical brain processes and activities than can potentiate the actions of psychedelics such processing supporting neurogenesis, synaptogenesis while blocking inflammatory cytokine activity particularly in the brain and potentiating the activity of anti-inflammatory cytokines.

### CROSS-REFERENCE TO RELATED APPLICATIONS

This application claims priority benefit from U.S. Provisional Application No. 63/261,711 filed on Sep. 27, 2021, U.S. Provisional Application No. 63/262,800 filed on Oct. 20, 2021, and U.S. Provisional Application No. 63/266,756 filed on Jan. 13, 2022. The patent applications identified above are incorporated here by reference in their entirety to provide continuity of disclosure.

### FIELD OF THE INVENTION

The emergence of psychedelics as potential treatments for mental health disorder raises the possibility of a new class of drugs that may be applicable across a broad spectrum of conditions. Psychedelics offer better efficacy, faster onset of action, longer duration of symptom remission and novel mechanisms of action over traditional treatments. For example, ketamine has recently been approved as a novel therapeutic for difficult-to-treat depression. However, three major challenges must be addressed to satisfy scientific and clinical standards for broader community adoption.

Blinding of treatment arms in psychedelic trials is a major challenge because the hallucinogenic state is typically obvious to the researcher(s) and subject. Non-blinding of the hallucinogenic state raises serious concerns about the validity and reliability of data produced in psychedelic clinical trials. For example, measured improvements may reflect the bias of the clinical researcher or subject who observed/experienced an obvious psychedelic episode with actual clinical improvement.

While an individual is high, they are in an extremely vulnerable, highly suggestive, and potentially psychotic (hallucinatory and delusional) state that can have major consequences following psychedelic treatment. The content of psychedelic trip is highly variable and difficult to manage. For example, a person during their psychedelic trip becomes convinced that they must leave their spouse and/or get a divorce after treatment (fixed delusion they would not have on their own results in permanent life altering decisions).

Other examples include a hallucinogenic experience inducing major world view and personality shifts that a person would not consider prior to psychedelic treatment. For example, it is often reported by users of DMT that “machine elves” are in some way controlling the environment. Exposure of a patient to the uncontrollable and unpredictable side effects of the hallucinogenic experience is considered by many clinicians to be unethical and too risky. Many consider the risk higher than the potential benefit of psychedelics as a viable treatment.

The current method of controlling for these safety objections are costly specialized treatment rooms/centers and expensive specially-trained therapists, and prolonged treatment and integration sessions. The logistical challenges of building a nationwide treatment network will be limited by the cost and availability of certified trained therapists. This makes current psychedelic treatment approaches untenable. The current invention addresses all three of these unresolved challenges, making psychedelics a viable treatment option.

### BACKGROUND OF THE INVENTION

There has been an immense evolution in the pharmacological treatment of mental health disorders since the introduction of chlorpromazine (marketed as Thorazine) for schizophrenia and psychosis in the 1950's and 1960's. The development of similar drugs such as chlorprothixene, thiothixene, and haloperidol, significantly reduced the incidence and severity of psychotic behaviors. The positive effects of these drugs on symptom severity facilitated a significance reduction in neuropsychiatric inpatient populations across the U.S. and worldwide as well. At least partly contributing to the efficacy of this first class of anti-psychotic medications was a strong sedation effect. Unfortunately, these original “typical” anti-psychotics have severe extra-pyramidal side effects, including tardive dyskinesia, producing involuntary movements of the face and jaw.

Further understanding of the neurological bases of mental health disorders expanded pharmacological treatment that include multiple brain areas and neurotransmitter systems. With schizophrenia, for example, theories of its origins and the resulting medication originally focused on dysfunction in the dopaminergic system. It was first posited that an excess of dopamine in the brain was related to severity of the schizophrenia and psychosis. Subsequent theories postulated hyperdopaminergic activity subcortically, and hypodopaminergic activity, cortically. Over the last 20 years, the evolution of the theories and treatment of mental health disorder origins advanced and expanded, most notably adding serotonin neurotransmitter system dysfunction as a target for depression, schizophrenia, and psychosis. More recent models have proposed that mental health disorders reflect other factors including elevation of neuroinflammatory immunological activity in the brain. Across all mental health disorders, suppression of neural mechanism associated with neurogenesis, synaptogenesis and neuroplasticity have also been implicated.

Among the existing pharmacological treatments currently under development for mental health disorders, psychedelic drugs are also under consideration. For many centuries, humans have used psychedelic drugs to induce states of altered cognitive function or consciousness for cultural and religious purposes.

With the onset of the United States' “war on drugs” in the early 1970's, almost all psychedelics became classified as Schedule 1 compounds, defined as having no known existing accepted medical use as well as a high potential for abuse. Harsh regulations and penalties for possession essentially eliminated research investigating the efficacy of these drugs for application in treating mental health disorders. However, over the past 20 years, there has been a dramatic increase in research investigating whether psychedelics have potential in understanding and in treating mental health disorders. A growing number of studies have demonstrated that psychedelic drugs have potential as treatments for a variety of psychiatric conditions.

A recently published clinical trial employing a psychedelic psilocybin-assisted psychotherapy for treatment-resistant major depression found that after two drug doses, large and sustained symptom reduction were found in 71% of participants that persisted at least 8 weeks after the intervention. This was a randomized trial, but blinding was restricted to the psychometric raters and did not include the patients or therapists. Therefore, separation of real drug effects from patient expectations and therapist contributions to clinical efficacy were difficult to separate. Importantly, a recent meta-analysis found that for depression, non-psychedelic medication, and psychotherapy, while significantly better than placebo when administered independently, show only marginal advantage when combined. As will be discussed later, appropriate blinding of the hallucinogenic experience is important, not just for the clinician/therapist, but also for the patient, to clearly demonstrate efficacy required for regulatory approval.

WIPO Publication No. WO 2020169851A to Terwey describes opinions regarding the mechanism(s) of psychedelic drug efficacy vary significantly. Some groups have focused almost exclusively on the importance of the hallucinogenic experience, associating efficacy with the intensity of the peak of the hallucinogenic experience of the psychedelic drug 5-MeO-DMT.

U.S. Publication No. 2020/0397752 to Perez Castillo, et al. focuses on the non-hallucinogenic effects that the same psychedelic, 5-MeO-DMT, produces on the underlying neurotransmitter systems, using a combined administration with a serotonin 5-HT2a receptor antagonist such as ketanserin, to block the hallucinogenic activity.

A significant issue for those focused on the hallucinogenic experience as the primary agent of efficacy is the immense variability associated with the cognitive and emotional content evoked during the altered state of consciousness produced by 5-MeO-DMT. Within the same individual, a psychedelic drug may induce immense feelings of joy and love in some sessions, while in other sessions provoke terror and panic in the same individual. For drugs such as 5-MeO-DMT, as specified in the preferred embodiment of this invention, hallucinations are extremely intense. Reported experiences include ego dissolution, or a compromised sense of self, accompanied by thoughts of imminent death, which can be followed by feelings of love and euphoria. Studies have demonstrated significant correlations between efficacy and perception of the hallucinogenic experience, but interpretation of these results are complex.

One investigation measured individual hallucinogenic experiences using a standardized psychometric scale for psychedelics. This scale included categories such as oceanic boundlessness, visual reconstructuralization, as well as dread of ego-dissolution and vigilance reduction. Results showed moderate but statistically significant correlations between these scales and scores on the Quick Inventory of Depressive Symptoms Scale. Individuals who experienced extremely high dread of ego-dissolution showed little/less improvement in depression scores while those who had high(emotionally positive) scores on the oceanic boundless measure had large improvements on the depression scale with statistical correlations as high as r=0.5. The results of this study and others with similar outcomes are important, but difficult to interpret.

First, the execution of psychedelic trials is frequently unblinded to both the patient and the clinician. In many of these studies, the lack of an effective placebo control is also common. For psychedelics, an absence of an effective method of blinding for both patient and clinician make it difficult if not impossible to include an effective placebo control. The conscious emotional impact of the initial acute hallucinogenic experience also has the potential to bias long-term expectations of outcome for both the patient and clinician.

Other investigators have pointed out that in unblinded studies with non-psychedelics, many confounding factors can contribute to a placebo response, including factors such as instillation of hope, response expectancies, motivation to please investigators, a therapeutic impact of clinical contact, rater bias and spontaneous improvement. Thus, interpretations of positive long term clinical outcomes being associated with more positive emotional perceptions of the transient hallucinogenic experience are subject to the same factors regarded as critical confounds observed in unblinded studies using non-psychedelic drugs.

The importance of maintaining effective blinding has been demonstrated in studies using psychedelic microdosing, which minimize or eliminate the hallucinogenic experience. One such study included more than 190 volunteer non-psychiatric participants who were recruited online. Results demonstrated a significant improvement in several psychometric measures including well-being, mindfulness, life satisfaction and paranoia between baseline and the week-four end-point of administration. However, similar magnitudes of improvement were also found for the placebo group as well. The investigators attributed the failure to find a significant active drug vs placebo effect to participants not being appropriately blinded.

Other studies evaluating the effect of initial positive expectations prior to study onset have concluded that microdosed psychedelics can act as active placebos which are predictive of clinical outcome. A review of previous psychedelic research concluded that in evaluating clinical efficacy, replacing placebo-controlled trials with non-inferiority trials significantly “lowers the bar of acceptance,” thus increasing the likelihood that medications for mental health disorders will be, “foisted on the public and, less dangerous but also problematic, that potentially effective agents would be missed”.

The objective of inferiority trials, according to the FDA (FDA-2010-D-0075), is to determine whether the efficacy of a test-drug is statistically not worse than an available treatment. Inferiority trials are most appropriately applied for evaluations of drugs known to prevent serious harm or death. Under such conditions, inclusion of a non-active placebo control would be regarded as morally and ethically inappropriate.

One stated criterion for allowing an inferiority trial design is for circumstances in which a standard accepted drug therapy is sufficiently toxic that many patients refuse its administration. Based on this criterion, the evaluation of efficacy for psychedelic drugs using an inferiority trial would not seem to quality for exemption. It therefore seems critical to develop novel methods and approaches that can allow blinding and employ appropriate placebo controls. Unfortunately, even when micro-dosing has been employed to minimize or eliminate the hallucinogenic experience, patient and clinician knowledge of the “novel” application of a psychedelic to treat a mental health disorder may be sufficient to modify expectations of outcomes, independent of any active drug effect. Clearly, subjective quality of a hallucinogenic experience is highly susceptible to contextual influences such as environment and expectations.

Terwey identified 5-MeO-DMT as part of the preferred embodiment to treat neuropsychiatric disorders such as depression. The novelty of this patent relies on a method that asserts conscious awareness of the hallucinogenic experience is essential and, critically, that attaining a peak psychedelic experience is also critical. The development of appropriate blinded, placebo-controlled trials for this patent would be difficult as both the patient and clinician are aware of the hallucinogenic experience. As previously indicated, such studies can introduce placebo effects associated with expectations, environmental effects, and suggestibility all related to the conscious experience of the hallucinogenic experience. Terwey also specifies that a “hallucinogenic peak” for 5-MeO-DMT be established in order to achieve optimal efficacy, which requires multiple dosing of this powerful psychedelic. It is important to note that under ostensibly naturalistic settings, “recreational” use occurs infrequently for psychedelics in the DMT family, including ayahuasca tea, DMT, and 5-MeO-DMT, typically less than once per year.

Even for one of the previously cited non-blinded trials in which a significant relationship between hallucinogenic experience (positive or negative) and efficacy has achieved correlations as high as r=0.5, the associated r² value of 25% indicates that only a minority of the explained variance can be attributed to the relationship between these variables. Explaining the remaining 75% of the variance in the relationship between psychedelics and clinical efficacy in treating mental health disorders therefore requires the consideration of other factors.

As discussed in Perez Castillo, targeting 5-Meo-DMT adopted a different approach. Perez Castillo effectively regards the hallucinogenic event and the associated brain activity as an unnecessary and confounding side effect. It specifies a method of a combined administration of the psychedelic with a second drug that that eliminates the hallucinogenic experience, specifically ketanserin (or another substantially equivalent selective 5-HT2a receptor antagonist). Traditional theories have argued that 5-HT2a receptor agonism is primarily or entirely responsible for hallucinations associated with psychedelics.

A psychedelic that produces intense hallucinogenic experiences such as 5-MeO-DMT is a 5-HT2a receptor agonist. Agonism of 5-HT2a receptor activity plays a significant role in inducing hallucinations. However, other neurochemical components of 5-MeO-DMT may also contribute toward inducing or inhibiting hallucinatory activity.

For non-psychedelic hallucinogens, such as dissociative anesthetics (e.g., ketamine or phencyclidine), hallucinations result by blocking glutamate receptors while psychostimulants (e.g., cocaine or amphetamine) induce hallucinations by activation of dopaminergic D2 receptors.

It is important to note that despite different receptor targets across different classes of hallucinogens, the content of the hallucinogenic experience is remarkably consistent. A canonical correlation analysis of more than 6800 written reports across a full range of psychedelic drugs indicates that ego dissolution is a common feature of the hallucinogenic experience across all classes of hallucinogens. Ego dissolution is regarded as hallmark of the 5-MeO-DMT hallucinogenic experience. However, results of the canonical analysis found that the ego dissolution experience occurs with all classes of hallucinogenic drugs. Thus, the ego dissolution effects of a serotoninergic hallucinogen such 5-MeO-DMT and NDMA receptor antagonists (such as ketamine) are qualitatively similar, despite major differences in the chemical structure of the drugs.

Importantly, non-serotonergic hallucinogens such as ketamine (i.e., NMDA-antagonist and dissociative anesthetic), the MDMA drug ibogaine, and salvinorin A (a kappa-opioid agonist), interact minimally, indirectly, or not at all with 5-HT2A receptors. This strongly suggests that a broader array of neurobiological mechanisms is involved in producing hallucinogenic activity.

Moreover, increasing evidence indicates that the subjective hallucinogenic effects of ketamine and serotonergic psychedelics might not be necessary for their sustained beneficial effects on neuroplasticity and behavior.

Singularly attributing hallucinations to the 5-HT2a receptor, alone, may therefore be an over-simplification. Using a 5-HT2a receptor antagonist such as ketanserin, as proposed in Perez Castillo, may only block a portion of hallucinogenic experience attributable to 5-HT2a agonism. Other features of the hallucinogenic experience may persist. More importantly, using ketanserin as a 5-HT2 receptor antagonist blocks other critical functions of this receptor that could adversely impact the efficacy of 5-MeO-DMT in treating mental health disorders.

U.S. Pat. No. 11,406,619 to Layzell, et al. discloses a pharmaceutical formulation suitable for injection, comprising: a salt of a dimethyltryptamine compound optionally substituted with deuterium and optionally substituted at position 4 or 5 with acetoxy or methoxy or position 4 with monohydrogen phosphate. The invention provides a method of treating a psychiatric or neurological disorder comprising administering to a patient in need thereof a formulation of the first aspect. The psychiatric or neurological disorder may be any of those described in relation to the sixth aspect. For example, the disorder may be selected from the group consisting of major depressive disorder, treatment resistant major depressive disorder, post-partum depression, an obsessive-compulsive disorder and an eating disorder such as a compulsive eating disorder.

WIPO Publication No. WO 2022082058A1 to Bridges, et al. discloses a method of treatment focused on tryptamines, the same classification as 5-Meo-DMT. A pharmacologically effective amount of tryptamine alkaloid is administered as a saline solution, this includes multiple tryptamines including 5-Meo-DMT. In certain cases, the infusion includes a pharmacologically effective amount of anesthetic or sedative. The psychological condition can be depression as well as anxiety, addiction, and PTSD.

U.S. Publication No. 2021/0023052 to Chadeayne discloses a purified toad secretion including 5-Meo-DMT and a second active serotonergic drug, a psilocybin derivative. The use for psychological disorders includes depressive disorder. No disclosure or suggestion of a possible anesthetic to modulate the effects of the active drug is made.

### SUMMARY OF THE INVENTION

This invention describes a method of psychedelic drug delivery that blinds both patient and clinician(s) to placebo/active drug administration, with coincident administration of a short-lasting anesthetic with or without a muscle relaxant agent to block conscious recall during the time period coinciding with the acute post-drug/placebo administration hallucinogenic experience or “trip”. Depending on the route of psychedelic administration (e.g., IV versus inhalation) the anesthetic will be administered before or after the psychedelic drug dosing. The purpose of the anesthetic is to produce a loss of conscious awareness (unconsciousness) and block recall in order to (1) blind the administrator and participant to treatment groups and (2) to control/block adverse hallucinogenic events. The invention specifies preferred anesthetics that target brain centers, neurochemical receptor systems, and functional processes maximally independent of those targeted by the psychedelic drug. In the preferred embodiment, the psychedelic drug 5-5-Methoxy-N,N-dimethyltryptamine (5-MeO-DMT) is administered with the short acting anesthetic, for example, propofol, to block conscious awareness of the hallucinogenic experience. Combining a psychedelic with a short “high” allows use of a short-lasting anesthetic, minimizing the risks of longer-term anesthesia for the subject. This treatment would be completed as an outpatient procedure, which can utilize the existing infrastructure of electroconvulsive therapy (ECT) clinics, addressing resource challenges addressed in (2).

The serotonergic psychedelic, 5-MeO-DMT was first synthesized in 1936 and later isolated from the indole alkaloids of the flower plant, dictyoloma incanescens. 5-MeO-DMT produces a short-duration, 5-20-minute, acute hallucinogenic state, primarily targeting serotonergic 5-HT1a and 5-HT2a, as well 5-HT2c and 5-HT7, receptors, as well as Sigma-1 receptors. Functionally, animal models have shown that the combined activity at 5-HT1a and 5-HT2a receptors produced by 5-MEO-DMT facilitates long-lasting neurogenesis and synaptogenesis in critical areas of the brain, in particular in the hippocampus resulting in increased brain plasticity, associated with improved learning and memory, Activation of sigma-1 receptors by 5-MeO-DMT facilitates potent immunoregulatory properties that increase the secretion level of anti-inflammatory interleukin 10 cytokine, and suppressing IFNc (Interferon-c) and granulocyte-macrophage colony-stimulating factor (GM-CSF) expression. Recent studies have shown that a common feature across neuropsychiatric disorders is an elevation of inflammatory immunoregulatory cytokines, which in the case of schizophrenia, are present at the time of the first episode of illness.

As part of the invention, there is a combined administration of the fast-acting anesthetic such as propofol that induces loss of consciousness to block memory formation and recall of the hallucinogenic experience. Propofol operates through a positive modulation of the inhibitory function of the neurotransmitter gamma-aminobutyric acid (GABA) through GABAa receptors. The combined administration of propofol, to induce a short-lasting period of unconsciousness that also suppresses recall, with 5-MEO-DMT provides a novel treatment for a broad range of neuropsychiatric diseases, allowing a blind control and inclusion of a placebo control as the core elements to this novel invention.

This method would be used to control blinding challenges by having all treatment arms including placebo receive the anesthetic, allowing scientists to separate real psychedelic drug effects from placebo effects.

The present invention provides a combination product for the treatment and/or prevention of psychiatric and other neurological disorders. The combination product comprises (1) a compound which promotes neurogenesis and has hallucinogenic and/or psychedelic side effects, and (2) and an appropriate anesthetic that suppresses and/or blocks conscious awareness and/or recall of the hallucinogenic and mind-altering thought content caused by the psychedelic drug. Further (3), the suppression and/or blocking of the hallucinogenic experience or “trip” addresses issues of moral and psychological consequences associated with a good or bad trip, and (4) allows for blinding of both subject and research professionals so that drug effects can be appropriately separated from placebo effects in a clinical research trial.

One aspect of the invention is combining administration of a consciousness-blocking anesthetic that will suppress awareness, consolidation, and recall of the hallucinogenic experience associated with the administration of an active psychedelic drug. This may or may not include a muscle relaxant. For drug efficacy trials, this combined administration would include the administration of a consciousness-blocking anesthetic with a placebo.

The method of administration of the anesthetic and psychedelic drug combination optimizes a state of anesthetically induced non-awareness during the hallucinogenic state and blocks post-anesthetic recall of the hallucinogenic experience for all routes of psychedelic and anesthetic administration or combination of such delivery methods. This method may include other alternative anesthetic/psychedelic drug delivery methods that optimizes the state of anesthetically induced unawareness and maximal psychedelic drug efficacy.

The combination of a consciousness blocking anesthetic with a psychedelic drug that facilities the maintenance of the blinded state of both the patient and the examiner, thus reducing potential bias/placebo effects by awareness of the placebo/active drug condition.

Another aspect of the invention comprises measuring the state of unconsciousness via abehavioral test intended to elicit a patient behavioral response or via a measure of brain activity based on a spontaneous or evoked electroencephalography as a biomarker of altered brain state associated with a loss of conscious awareness. The measurement of brain activity may include both time and frequency domain measurements to detect awake, iso-potential, burst, and burst suppression EEG states in real or near-real time.

In yet another aspect of the invention, the administration of a consciousness-blocking agent that will suppress consolidation and recall during the hallucinogenic experience or any other adverse effects such as heightened suggestibility associated with this state, to protect both the patient and the medical staff from intentionally or unintentionally adversely creating thoughts, beliefs, attitudes, emotions, and/or memories in the patient that could impact future, life choices, well-being, and/or quality of life.

The consciousness-blocking anesthetic is from a group of anesthetics (preferred embodiment, Diprivan, also known as propofol, or Sevorflurane, also known as fluormethyl, isoflurane or other similarly acting fast anesthetics) that minimally impact the function of serotonin receptors, primarily, but not restricted to the 5-HT1a and 5-HT2a serotonin receptors as well as Sigma-1 receptors.

The selection of a preferred psychedelic drug is based on the duration of its induced hallucinogenic state, with a preference for those drugs that produce shorter rather than longer hallucinogenic states, such as but not restricted to 5-MeO-DMT (e.g., T, 5-MeO-AMT, 5-MeO-DET, 5-MeO-DALT, 6-MeO-isoDMT, and taberanthalog).

In another aspect of the invention, the preferred psychedelic drug shall have the properties of altering activity at serotonin receptors, primarily, but not restricted to 5-HT1a and 5-HT2a receptors, as well as Sigma-1 receptors, such as but not restricted to 5-MeO-DMT.

In another aspect of the invention, the psychedelic drug will have among its properties, evidence increasing neurogenesis and synaptic plasticity, reducing over the long term, the production of inflammatory cytokines such as but not restricted to IL-1 and IL-6, and increasing the production of anti-inflammatorycytokines, such as IL010, as well as BDNF, such as but not restricted to 5-MeO-DMT.

The combined administration of the selected anesthetic and psychedelic drug with or without a muscle relaxant will target mental health disorders. This invention also applies to the treatment of tachyphylaxis which reflects a rapidly diminishing response to successive doses of a drug, a common issue with drugs acting on the nervous system or elsewhere in the body.

The intended use of the combined treatment will also extend to cognitive/self enhancement for individuals not affected by mental health disorders. Cognitive enhancement is defined, but not restricted to alteration or improvement in emotional, intellectual, or creative status.

The intended use of the combined treatment will also extend to treatment of autoimmune disorders. The intended use will also include phantom sensory phenomena including but not restricted to tinnitus and phantom limb syndrome as well as chronic pain.

### BRIEF DESCRIPTION OF THE DRAWINGS

In the detailed description of the preferred embodiments presented below, reference is made to the accompanying drawings.

FIGS. 1A and 1B is a flowchart of a method for treating mental health disorders.

### DETAILED DESCRIPTION OF THE INVENTION

As previously discussed, recent theories argue that mental health disorders are complex processes including multi-neurotransmitter systems and receptor types, (dys)modulation of neuro- and synapto-genesis necessary for neuroplasticity, and immunological overactivation resulting in elevated levels of neuroinflammatory activity within the central nervous system. The drug 5-MeO-DMT and other similar psychedelics (including 6-MEO-isoDMT, and Tabernanthalog, as well as others) are structural analogs of serotonin and melatonin as well as a functional analog of other psychedelic tryptamines.

5-MeO-DMT has high affinity for serotonin receptor types, 5-HT1a, 5HT2a and 5HT2c, with lessor affinity for other serotonin receptors, including 5-HT1b, 5-HT1d, 5-HT6 and 5-HT7. In addition, 5-MeO-DMT also has a functionally significant affinity for Sigma-1 receptors that modulate immune responses in human monocyte-derived dendritic cells. Originating in the midbrain raphe nuclei, serotonergic neurons account for 5-HT release at terminal nerve ends throughout the brain. In the human raphe nuclei, between 80-100% of 5-HT1a receptors are located on cell bodies of serotonin neurons and perform autoinhibitory functions.

Activation of 5-HT1a auto receptors consequently reduces the rate of serotonergic cell firing resulting in reduced serotonin release. Outside the raphe nucleus 5-HT1a receptors are mainly situated on non-serotonin-containing neurons in projection areas, function as heteroreceptors which inhibit postsynaptic cell firing. 5-HT2a receptor are G-protein couple receptors, which have been identified in the dorsal raphe nucleus and are present throughout many cortical areas, notably prefrontal cortex where a substantial portion of excitatory pyramidal neurons express 5-HT2a receptor mRNA.

While primarily an excitatory receptor type, it may have inhibitory effects on specific areas of the brain including visual and orbital frontal cortex. In the cynomolgus monkey, Sigma-1 binding potential was highest in the amygdala, hippocampus, insula, and frontal cortex. A number of investigations have demonstrated a role for Sigma-1 receptor activity in a number of neuropsychiatric disorders including Alzheimer's disease depression, chronic pain, and drug addiction

Combined, studies of 5-MeO-DMT (as well as other typical and atypical psychedelics) show robust evidence of serotonin receptors, primarily 5-HT1a and 5-HT2a, in modulating neural activity identified as contributing to normal or dysfunctional mental health. Serotonin system dysfunction has been linked with several major mental disorders including depression and schizophrenia. Thus, the application of so-called typical serotonergic psychedelics to alter serotonergic function in the brain has face validity. Clinical efficacy of serotonergic psychedelics mostly reflects the dense and widespread expression of these 5-HT receptors throughout human brain, both cortically and subcortically.

Recent research using human cerebral organoids induced from embryonic stem cells revealed that the administration of 5-MeO-DMT has a number of effects regarded as beneficial in treating mental illness. These beneficial effects include a potentiation of neural plasticity and elevated anti-inflammatory activity. This research demonstrated that just a single dose of 5-MeO-DMT modulates activity in signaling molecules critical in long term potentiation (LTP) indices of learning and memory. LTP is defined as a long-lasting (>24 hours) strengthening of postsynaptic neural responses to stimulation across the synapse that occurs following a brief period of rapidly repeated electrical stimulation. Long-term potentiation of synaptic transmission in the hippocampus is the leading experimental model for the synaptic changes that may underlie learning and memory. Dendritic spine morphogenesis proteins and cytoskeletal reorganization are also altered by 5-MeO-DMT, which are key elements of neural plasticity. The administration of 5-MeO-DMT also resulted in a downregulation of mGluR5, which plays a vital role in the rewarding effects for several commonly abused drugs such as cocaine.

Research indicates that the anti-inflammatory effects of 5-MeO-DMT act through modulation and downregulation of Gq-coupled protein receptors, mostly representing 5-HT2a (and perhaps 5-HT2c) receptor activity. Over the past two decades, a high comorbidity between autoimmune conditions and mental and mood disorders, such as MDD, anxiety, schizophrenia, psychosis, and bipolar disorder has become apparent.

For example, those individuals diagnosed with an autoimmune condition have a documented higher risk of developing clinical depression or mood disorders. Autoimmune conditions could contribute to MDD via dysregulated inflammatory cytokine loops between peripheral and brain-resident immune cells. Psychedelics, such as 5-MeO-DMT and other similar compounds impact immunomodulatory activity through the mobilization of cell-intrinsic neuroprotective mechanisms. Thus, in addition to treating mental health disorders, psychedelics can potentially provide a novel treatment for autoimmune-related disorders and associated mental illnesses.

Accumulating evidence indicates that the 5-HT2a receptor mediates many critical aspects of brain function and behavior, beyond generating hallucinations. 5-HT2a receptor agonism leads to a strong, glutamate-dependent increase in the activity of cortical pyramidal neurons, preferentially for those in layer V of the prefrontal cortex (PFC).

Recent studies indicate that stimulation of postsynaptic 5-HT2a receptors work on subpopulations of pyramidal cells found in deep PFC layers by increasing activity within a recurrent glutaminergic network linking prefrontal and limbic structures. Existing models of emotional regulation include both the PFC as well as the anterior cingulate cortex acting in concert to exert top-down (i.e., cognitive) control over emotional and stress responses via connection to sub-cortical limbic structures such as the amygdala as well as the dorsal raphe nucleus. Reduced prefrontal glutamate levels associated with reduced PFC activation is regarded as contributing significantly to major depressive disorder.

In rat models, the NMDA dissociative anesthetic ketamine as well psilocybin increases extracellular glutamate levels in prefrontal-limbic circuitry. In depressed human patients, the clinical efficacy demonstrated for both ketamine and psilocybin (which is structurally similar to 5-MeO-DMT) significantly outlast the acute hallucinogenic experience.

Functionally, studies in animal models have shown that via its actions on pyramidal cells and GABAergic interneurons, 5-HT2a receptor activity also regulates synaptic plasticity. Synaptic plasticity and neural plasticity in general, are defined as the ability to establish a relatively long-lasting change in activity/behavior due to prior experience. For 5-HT2a receptors, application of an antagonist that blocks activity at this receptor results in LTP deficits. Conversely, application 5-HT2a receptor agonists have the opposite effect. Thus, 5-HT2a receptors play a critical role beyond that resulting in hallucinations.

Via 5-HT2a agonism, 5-MeO-DMT also activates second messenger pathways including a differentially strong phospholipase A2 (PLA2) activation. PLA2 regulates proinflammatory responses in the brain. An imbalance in PLA2 regulation results in a disproportionate increase in proinflammatory agents.

It has been argued that the resulting neuroinflammation and oxidative stress plays a significant role in several brain disorders including Alzheimer's disease, schizophrenia, autism, major depressive disorder and ADHD. Moreover, PLA2 inhibitors block AMPAR-associated LTP, while increasing PLA2 activity enhances LTP. In rats it has been shown that a general inhibition of brain PLA2 activity decreases learning and memory formation.

In recognition of these findings, recent theories contend that serotonin differentially encodes behavioral and physiological responses to uncertainty. More specifically, limbic-rich postsynaptic 5-HT1a receptor antagonists provide basal control of behavior during normal conditions, by moderating emotion and anxiety, thus inducing a state of calm. In contrast, the cortically rich 5-HT2a receptors are differentially active during conditions of crisis, in essence, reflexively triggering the fight or flight responses as required.

Applying a Bayesian approach, this model suggests that the principal function of 5-HT2a receptor stimulation is a relaxation of prior assumptions or beliefs, held at multiple levels of the brain's functional hierarchy: perceptually, emotionally, cognitively, and philosophically (e.g., in terms of biases). Essentially, 5-HT2a receptor agonism facilitates heightened sensitivity to different perspectives or possibilities, which likely reflects elevated functional plasticity facilitating emotional change.

Consequently, removing or suppressing normal 5-HT2a receptor activity with an antagonist such as ketanserin, as discussed in Perez Castillo, can significantly alter the clinical efficacy of psychedelics such as 5-MeO-DMT, that are essential for therapeutic effectiveness including neuroplasticity, neurogenesis, neurodevelopment, learning, extinction learning, cognitive flexibility and enhanced environmental sensitivity.

In vitro preparations have shown that a single dose of 5-Meo-DMT enhances neurogenesis in the ventral dentate gyms of adult mice, presumed to reflect activation of 5-HT2a (and possibly 5-HT2c and 5 HT1-a) receptors. Thus, 5-HT2a receptor performs a number of critical, non-hallucinogenic functions, including modulation of glutaminergic dependent fronto-cortical limbic system networks essential in regulating mental health.

Overall, the widespread presence of 5-HT2a receptors on layer V pyramidal cells and the functional significance of these cells on cortico-limbic activity are staunch support for the role that 5-HT2a receptor selective agonists perform in controlling cortical feedback in a dose dependent manner. 5-HT2a receptor signaling via kalirin-7 signaling also influences synaptic structural plasticity in cortical pyramidal neurons resulting in a rapid modulation of dendritic spine morphology which can play a key role in neuropsychiatric disorders.

The combined activity of 5-HT2a and 5-HT1a receptors also have an important impact on brain activity by acting in concert to modulate neuronal membrane excitability through opposing effects. Most pyramidal neurons express both receptor subtypes on their membrane surfaces. Studies have shown that co-activation of both receptor subtypes potentiate responsiveness to strong excitatory stimuli while having negligible impact on weaker stimuli. As a result, the presence of the two serotonin receptors that mediate opposite effects on membrane excitability in the same cell provides an adaptable mechanism in encoding pyramidal activity evoked by excitatory input.

Dorsolateral prefrontal cortex networks are regarded as critically important in working memory processes essential for normal human cognition. Deficiency in these processes is associated with the negative symptoms and cognitive deficits that are commonly observed in schizophrenia as well as positive symptoms of this disorder.

The combined effects of 5-HT2a and 5-HT1a receptor activity alter brain activity in other ways. The raphe nucleus in the brainstem is largely responsible for the release of serotonin in the brain. The dorsal Raphe nucleus (DRN) is the principal source of serotonergic innervation for the cerebral cortex, amygdala, BFB, thalamus, preoptic and hypothalamic areas, other Raphe nuclei, the locus coeruleus, and pontine reticular formation. In particular, prefrontal cortex is a target of substantial innervation by serotonergic fibers from the dorsal raphe nucleus in both primates and humans. Intracellular recordings from human frontal cortical slices show that neurons can be depolarized via 5-HT2a receptors and hyperpolarized via 5-HT1a receptors.

In addition, inhibitory and excitatory neurotransmitter system projections to the dorsal raphe nucleus regulate the activity of 5-HT1a and 5-HT2a receptors during the sleep wake cycle. Selective activation of somatodendritic 5-HT1a receptors in the dorsal raphe induces an increase of REM sleep while drugs that antagonize 5-HT2a (and/or 5-HT2c) receptors enhance slow wave sleep. The combined effects of 5-HT1a and 5-HT2a receptor activity on sleep are critically important for mental health disorders as altered sleep structure is documented for almost all mental health disorders. Improved symptom severity for disorders including depression and schizophrenia is associated with the normalization of sleep treated with medications that include modulation of serotonergic system activity. Thus, disrupted sleep architecture is no longer regarded as just a symptom of mental health disorders, but can be causative as well.

The interactive relationship between the serotonergic receptors 5-HT2a and 5-HT1a and brain developmental neurotrophic factor (BDNF) can play a critical role in the efficacy of 5-MeO-DMT in treating mental health disorders. BDNF functions as a major regulator of activity-dependent plasticity at excitatory synapses through the central nervous system including, importantly, the hippocampus. The hippocampus has an essential role in the formation, organization, and storage of new memories as well as connecting certain sensations and emotions to those memories.

According to a recent review, the combined activity of the serotonin and BDNF systems underlies mechanisms of brain plasticity that regulate normal and pathological behaviors. For example, targeted disruption of BDNF in the mouse hippocampus results in a significant impairment of LTP in hippocampal CA1 synapses, indicating neuroplasticity dysfunction. A single dose of 5-MeO-DMT stimulates cell proliferation and neuronal survivability in the ventral dentate gyrus of the hippocampus, as well as generating other morphological and functional changes, leading to the conclusion that 5-HT1a and 5-HT2a receptor targets modulate neurogenesis in multiple areas of the brain including the hippocampus and the subgranular portion of the ventral dentate gyrus, where neurogenesis is maintained through adulthood.

Vitally important, this relationship between BDNF and the 5-HT systems is reciprocal, with interactions affecting each system's activity. The serotonin receptors 5-HT1a and 5-HT2a and BDNF share common signal pathways and have similar secondary messengers, or molecules inside cells that acts to transmit signals from a receptor to a target. In rats, acute and chronic BDNF administration into the lateral ventricle increases serotonin activity (including 5-HT1a and 5-HT2a receptors) in numerous brain structures including the frontal cortex, hippocampus, striatum, hypothalamus, substantia nigra and nucleus accumbens.

Moreover, BDNF knock-out mice show prominent postsynaptic 5-HT2a receptor deficits in the prefrontal cortex and dorsal raphe nucleus along with a virtual absence of normal 5-HT2a-mediated glutamate and GABA postsynaptic potentials in the prefrontal cortex. Thus, impaired BDNF expression results in overall serotonergic system dysfunction, expressed as reduced serotonin transporter activity (responsible for re-uptake of serotonin) and 5-HT1a receptor function in the hippocampus, as wells as severe 5-HT2a receptor deficits in the prefrontal cortex and dorsal raphe nucleus.

Conversely, for rats exposed to psychological stress, administration of 5-HT1a and 5-HT2a agonists similar to 5-MeO-DMT (specifically DPAT-PS and DOI-PS) upregulate immunoreactivity indicative of BDNF presence in the prefrontal cortex, the CA1 and CA3 regions of the hippocampus, as well as the prefrontal cortex, central amygdaloid nucleus, dorsomedial hypothalamic nucleus, dentate gyrus, ventral tegmental area, the shell of the nucleus accumbens and the midbrain periaqueductal gray. In these same brain areas, the administration of a 5-HT1a and 5-HT2a antagonist, ketanserin, inhibits BDNF immunoreactivity and, at the same time, reducing the presence BDNF mRNA. As mentioned previously, ketanserin is used to block the hallucinogenic effects of psychedelic and other dissociative drugs such as ketamine. Ketanserin, and similar 5-HT2a antagonists, are proposed as the drugs to block the hallucinogenic activity generated by 5-MeO-DMT as disclosed in Perez Castillo.

Clinically, disruption of the 5-HT-BDNF system interaction results in increased depressive disorder, suicidality, and aggressiveness. Results of a meta-analysis of previous studies indicate that increases in BDNF levels are statistically correlated with improved depression scores. Overall results show that elevated BDNF levels are associated with improvements in depression, supporting the idea that depression and its treatment are associated with neuroplastic changes associated with modulation of the 5-HT-BDNF systems. Research consistently demonstrates that BDNF promotes the survival and differentiation of serotonergic neurons. Further, selective serotonin reuptake inhibitors (SSRIs) enhance BDNF gene expression. Collectively, there is unmistakable evidence that a synergism exists between these two systems in modulating affective behavior and mental health.

Independently, 5-HT1a receptor activity also plays a critical role in neuronal development and plasticity particularly in the hippocampus. The 5-HT1a receptor represents one of the most abundant serotonin receptor subtypes expressed in the mammalian brain. In humans, Positron Emission Tomography (PET) binding potential methods have shown that 5-HT1a receptor density is highest in hippocampal regions, insula, neocortical regions, and dorsal raphe nucleus, and much lower in the thalamus and basal ganglia. The majority of PET studies have confirmed alterations of the 5-HT1A receptor binding in patients demonstrating widespread reductions of 5-HT1a receptor binding in depressed and anxiety disorders patients compared to non-depressed control subjects.

Activity associated with 5-HT1a receptors serves many functions. The locations and actions of 5-HT1a receptors, both pre- and post-synaptically, are strongly correlated with brain areas associated with emotional and affective processes. Presynaptic 5-HT1a receptors regulate autoinhibition of serotonergic firing and release are located on serotonergic neurons in the midbrain raphe region. Post-synaptically, 5-HT1a receptors mediate inhibition and excitation that is mainly expressed on glutamatergic and GABAergic neurons. Post-synaptic 5-HT1a receptor activity can also induce widespread inhibition of serotonergic cell firing, indicating that postsynaptic 5-HT1a receptors participate in the autoregulation of the entire brain serotonergic system.

Clinically, a large body of research has shown that abnormal 5-HT1a receptor activity is involved in a number of neuropsychiatric disorders including schizophrenia, major depression disorder and anxiety, suicide, substance abuse and drug addiction, Parkinson's disease, Alzheimer's disease as well post-traumatic stress disorder and traumatic brain injury. 5-HT1 1a receptor activity as well as other serotonin receptors also play a role in modulating immune system activity.

Individually and combined, clinical research has reinforced the role of 5-HT1a and 5-HT2a receptor activity inherent to the actions of psychedelics such as 5-MeO-DMT in alleviating the severity of mental health disorders. Collectively, activity at these receptors is critical in modulating many neural processes essential for maintaining brain equilibrium. In addition to its actions involving these critical serotonin receptors, 5-MeO-DMT also significantly potentiates activity for at least one other receptor; the Sigma-1 receptor, which like serotonergic receptors, also contributes to modulating neuroinflammatory activity. The Sigma-1 receptor ligands (molecules) contain strong immunoregulatory properties that increase the secretion of anti-inflammatory agents such as interleukin-10 (IL-10) and suppress human interferon gamma (IFNγ) and granulocyte-macrophage colony-stimulating factor (GM-CSF) expression.

Upregulation of these agents potentiates many physiological processes including cell proliferation and survival throughout the body, as well as the brain. Sigma-1 has also been detected in immune cells mediating strong immunosuppressive and anti-inflammatory effects. Potentiation of anti-inflammatory responses can contribute to the efficacy of psychedelics such as 5-MeO-DMT as it has been shown that elevated levels of pro-inflammatory cytokines are associated with many mental health disorders including obsessive compulsive disorder, major depressive disorder, anxiety disorder, schizophrenia, psychosis, Alzheimer's disease, chronic pain and substance abuse, including alcoholism.

Studies in mice have also shown that tinnitus, which can be of peripheral or central origin, can be reduced by a single dose of 5-Meo-DMT. Many studies have investigated changes in central nervous system activity associated with tinnitus, implicating frontal cortex and limbic areas. 5-MeO-DMT, via 5-HT1a and 5-HT2a receptor activity, can modulate tinnitus intensity and emotional responses to this persistent perception of noise or tonal sound. It has also been argued that psychedelics such as 5-MeO-DMT, primarily through modulation of 5-HT2a receptor activity, offers a novel treatment approach for chronic and phantom pain disorders.

Finally, recent studies indicate that the 5-HT7 receptor, for which 5-MeO-DMT also shows binding activity, may be important as a potential target for treating alcoholism and other addictions.

Taken together, the sum of current research indicates the clinical efficacy of psychedelics like 5-MeO-DMT is dependent on all facets of its chemical structure and its impact on brain activity. Suppressing or eliminating the hallucinogenic experience by administration of 5-HT2a antagonist also blocks activity of this serotonin receptor that has critical role in the efficacy of 5-MeO-DMT.

The present invention maintains 5-HT2a receptor function but blocks conscious awareness and recall of the hallucinogenic experience with the combined administration of an appropriate anesthetic. In the preferred embodiment, the chosen anesthetic interacts minimally with 5-MeO-DMT on brain systems and function regarded as critical to the clinical efficacy of this psychedelic.

In addition, the chosen anesthetic is fast-acting and short-lasting, so that the duration of unconsciousness corresponds with the short duration of the 5-MeO-DMT hallucinogenic state. Third, the anesthetic specifically blocks conscious awareness and recall of the hallucinogenic experience. Fourth, the selected anesthetic is established as having either minimal effect or the potential to enhance clinical efficacy in combined administration for the treatment of mental health disorders including major depressive disorder. For the preferred embodiment, propofol is identified as the anesthetic that satisfies these criteria.

Propofol is an intravenous anesthetic that functions by increasing GABA-mediated inhibitory tone in the CNS. This decreases the rate of dissociation of GABA from the receptor, thereby increasing the duration of the GABA-activated openings of chloride channels with resulting hyperpolarization of cell membranes which results in a loss of consciousness. Acutely, propofol inhibits long term potentiation (LTP) induction through modulation of gamma-aminobutyric acid type A receptors but, unlike ketamine, does not induce inhibition of N-methyl-D-aspartate (NMDA) receptors.

LTP, as previously indicated, is critical to memory and learning. The acute inhibition of LTP by propofol is the process by which conscious awareness and recall are blocked. These short-term modifications of GABA function in the brain by propofol are largely independent of those for 5-MeO-DMT which mostly operates through serotonin and Sigma brain receptors. High doses of propofol induce burst-suppression, fundamentally inhibiting the state of intrinsic cortical hyperexcitability that defines burst activity. Burst suppression, quantifiable using electroencephalography (EEG) is disrupted by blockade of GABA.

Animal studies have shown that the administration of propofol in a sub-anesthetic dose can decrease the speed and degree of the glutamate-induced desensitization of the AMPA receptor in neurons and increase the expression of AMPA pGluR1 in dose-dependent manner. The upregulation of AMPA receptor GluR1 subunit phosphorylation provides evidence supporting the AMPA receptor as a molecular target for this anesthetic. Compounds that augment signaling through AMPA receptors (AMPA receptor potentiators) exhibit antidepressant-like behavioral effects in animal models. The resulting impact on neuronal function akin to those produced by several existing antidepressants. This includes neurotrophin induction along with increases in hippocampal progenitor cell proliferation. AMPA receptors also regulate the expression of BDNF, which contribute to the efficacy antidepressant drugs. Combined, these data suggest that AMPA receptors can regulate cortical processes such as BDNF expression and neurogenesis related mood disorders.

Importantly, propofol does not diminish the efficacy of ECT in treating depression. A direct comparison of Propofol and ECT demonstrated comparable outcomes in the reduction of self-reported depression symptoms using the 16-item Quick Inventory of Depressive Symptomology (QIDS-SR) scale. Propofol dosing in this investigation is based on a maintaining a suppression ratio (SR) of at least 80% (reflecting the fraction of time the EEG is isoelectric and free of bursting activity during each 1-minute epoch) over a period of 15 minutes.

Upon induction with propofol, subjects typically entered a state of unconscious within one minute. About five minutes after induction, the desired SR>80% was reached, and maintained for 10 to 17.5 minutes. Once propofol was discontinued, participants remained unconscious with eyes closed for 20 to 47 minutes, followed by eye opening, and an average recovery time in which the patient became oriented to time and place of approximately 5-10 minutes. Propofol treatment sessions occurred three times per week continuing for a total of ten sessions.

A similar treatment schedule was maintained for the ECT group. Results showed a significant reduction in QIDS-SR scale scores after approximately four sessions (1 week) and continued to improve before asymptoting at approximately 7 sessions (two weeks). Comparable results were obtained with ECT, although asymptotic scores on the QID-RS scale showed a consistent trend to more improvement for propofol than for ECT. Importantly, the cognitive side effects common for ECT (short term memory loss) do not occur with propofol treatment.

The results of this generate important conclusions. First, propofol acts independently of the serotoninergic neurotransmitter systems while resulting in loss of consciousness and recall during the anesthetic state. Acutely, Propofol blocks LTP, which as described previously, is critical in the formation of memories and learning. Results from animal studies indicate that the process by which conscious awareness and recall are blocked is related to the effects of propofol on LTP. Second, preliminary evidence exists that propofol can reduce the severity of self-report symptoms of moderate to severe depression within a period of 1-2 weeks with a pattern and degree of improvement comparable to that produced by ECT.

The scope of the claims are not restricted to or limited by the preferred embodiment. The most significant dangers of psychedelics lie principally in their psychological effects, with the ability to induce positive and negative perceptual and cognitive effects as well as emotional and autonomic responses typically only observed with psychoses. In uncontrolled and so-called natural settings conditions, the use of psychedelics such LSD, ayahuasca, psilocybin, DMT and 5-MeO-DMT include an array of psychedelic experiences often including intense fear which can temporarily debilitate the individual and/or lead to violent suicidal or homicidal ideas or behavior triggered by panic. Moreover, it has been shown that hallucinogenic experience can aggravate underlying psychiatric conditions including schizophrenia, bipolar disorders, and anxiety disorders.

While it seems contradictory, psychedelics, when carefully and appropriately engineered, manufactured, targeted, and administered, can facilitate almost immediate and prolonged improvement of symptom severity associated with mental health disorders such as drug-resistant severe depression. At present, the apparent rapid reversal of symptom severity and prolonged maintenance of relief for severe depression by psychedelics is paralleled only by electroconvulsive therapy. Results from clinical studies using psychedelics have been interpreted by some as indicating that the key component to efficacy specifically relates to the psychological effects of being consciously aware during the hallucinogenic experience.

However, the hallucinogenic experience predominately reflects that action of psychedelics of agonistic activity of one neurotransmitter receptor, 5-HT2a and its effects on predominately frontal lobe activity. For psychedelics drugs, this hallucinogenic activity represents just one of a much larger group of neural processes impacting brain activity both cortically and subcortically. An examination of the growing scientific and clinical literature on psychedelics based not just on humans, but animal models, demonstrates complex and multi-faceted effects on both the brain and the autonomic nervous system.

The development of this preferred embodiment reflects these complex and multi-faceted actions on both central and autonomic nervous system activity. The preferred embodiment also incorporates a combined delivery administration approach that facilitates necessary blinding and placebo control to evaluate efficacy within the bounds of traditional regulatory practices. The specified blinding control gained by anesthetic sedation to induce unconsciousness and block recall, also serves to protect the patient from a heightened state of suggestibility as well as negative affective and/or cognitive experiences induced during the hallucinogenic experience.

The factors guiding the development of the preferred embodiment are (1) the selection of a powerful psychedelic demonstrated to have (2) a rapid and (3) long-lasting impact on symptom severity of mental health disorders. This is not intended to limit these claims to a specific number of treatment sessions and/or dosages.

The psychedelic specified in the preferred embodiment will have the functional characteristics of rapidly reversing the presence of inflammatory cytokines in the brain, while potentiating the activity of anti-inflammatory cytokines, supporting neurogenesis and synaptogenesis, thus facilitating a period of increased and prolong neural plasticity, which support learning and memory.

The preferred embodiment utilizes psychedelics that induce the shortest possible hallucinogenic experience, to facilitate the use of fast-acting, short duration anesthetics that creates a state of unconsciousness that eliminates and/or minimizes conscious awareness and/or recall of the hallucinogenic experience. This combination minimizes risks associated with a prolonged hallucinogenic state requiring a prolonged state of anesthesia. This state of unconscious induced by the fast-acting anesthetic can be monitored in real or near real time based on the spectral signature of the EEG. A fast-acting anesthetic will be employed that eliminates or minimizes conscious awareness will act on brain mechanisms and neurotransmitter systems that are maximally independent of those involved in the actions specified to the selected psychedelic drugs, and if possible facilitate critical brain processes and activities than can potentiate the actions of psychedelics such processing supporting neurogenesis, synaptogenesis while blocking inflammatory cytokine activity particularly in the brain, and potentiating the activity of anti-inflammatory cytokines.

The psychedelic drug specified, but not restricted to, is 5-MeO-DMT, which satisfies the criteria of a very intense (10 time stronger than LSD), short lasting (10-20 minutes) hallucinogenic experience that produces rapid and long-lasting reduction in symptom severity demonstrated primarily for drug-resistant major depressive disorder. The fast-acting and reversible anesthetic propofol (and other similar anesthetics) is selected for inducing an unconscious state that blocks conscious awareness and recall of the hallucinogenic state. Propofol (or other similar anesthetic) is selected as its actions are largely independent of brain areas and neurotransmitter systems activated by 5-MeO-DMT. Moreover, propofol and similar anesthetics show evidence of rapid suppression of neuroinflammatory cytokines. In addition, post-anesthetic effects show potentiation of hippocampal activities supporting learning and memory.

Monitoring of the state of unconsciousness induced by propofol will be in near real time or real time using EEG to identify an established signature of propofol presents for induced unconsciousness that is characterized by a suppression of neural activity above 20 Hz, that is dominated by intense brain activity concentrated in the high alpha/low beta frequency range (12-15 Hz), also referred to as the Sigma band in sleep EEG, and an elevated low frequency band between 0.1 and 2 Hz. The preferred embodiment will employ an ambulatory EEG device (e.g., Medtronic Bispectral Index (BIS) Complete 2-Channel Monitoring System for depth of anesthesia) with electrodes placed on the forehead to that converts the time-domain EEG activity into the frequency domain via a fast Fourier or a wavelet analysis which is displayed on a hand-held smart device such as a tablet or cellular phone, or on a computer.

Identification of the spectral signature associated with unconsciousness and the loss of conscious awareness and recall will be automatically detected in the preferred embodiment but can also be identified visually by a professional trained and skilled in the art of EEG analysis. The intended use of the preferred embodiment combining psychedelic drug or drugs with anesthesia with or without muscle relaxant for evaluation of drug efficacy in double blind clinical trials and the treatment of neuropsychiatric illness (e.g., depressive disorder, bipolar disorder, obsessive compulsive disorder, autism, attention deficit hyperactivity disorder, psychosis, schizophrenia, Alzheimer's disease and other disorders), substance use disorders, tachyphylaxis, and personal cognitive enhancement. Cognitive enhancement is described but not restricted to an alteration or improvement in emotional, intellectual, or creative status. This intended use of the combined treatment will also extend to cognitive/self enhancement for individuals not affected by mental health disorders.

The invention also has potential application for the treatment of tinnitus, which centrally consists of a sensory component (the sound) and often a strongly negative emotional component, which appears to be result in abnormal amygdala and frontal cortex activity. Moreover, considering recent research, the invention also applies as a potential treatment for autoimmune disease. While the preferred embodiment includes psychiatric and pain medications, the intended use is not restricted to these medication classes.

The invention specifically targets a combined administration, first, of a consciousness-blocking anesthetic (e.g., propofol, isoflurane, or sevoflurane in the preferred embodiment) or similar anesthetics that minimally impacts serotonin receptors, primarily the 5-HT1a and 5-HT2a receptors (and the 5-HT7 receptor), as well as the Sigma-1 receptors, followed by the administration of a psychedelic drug (in the preferred embodiment, 5-MeO-DMT, as well as but not restricted to 5-MeO-DALT, 6-MeO-isoDMT, and taberanthalog) such that the patient has minimal or no conscious recall of the hallucinogenic experience. For clinical drug trials, the anesthetically derived state of conscious unawareness facilitates maintenance of blinding both the patient and the clinician to the active drug/placebo treatment status, thus facilitating a more unbiased evaluation of efficacy.

Moreover, maintenance of conscious unawareness during the hallucinogenic phase also minimizes adverse reactions like intentional or inadvertent alteration of patient's thought processes by external environmental input (including the clinician) during this heightened state of suggestibility. This combination of a conscious awareness-blocking anesthetic combined with administration of the psychedelic drug (not limited to a specific order) is intended to minimize recall of hallucinogenic “trip,” while retaining all other aspects of the psychedelic drug's unique capacity to target and alter critical brain areas and functions that result in a rapid reduction of the severity of symptoms associated with the mental health disorder experienced by the patient.

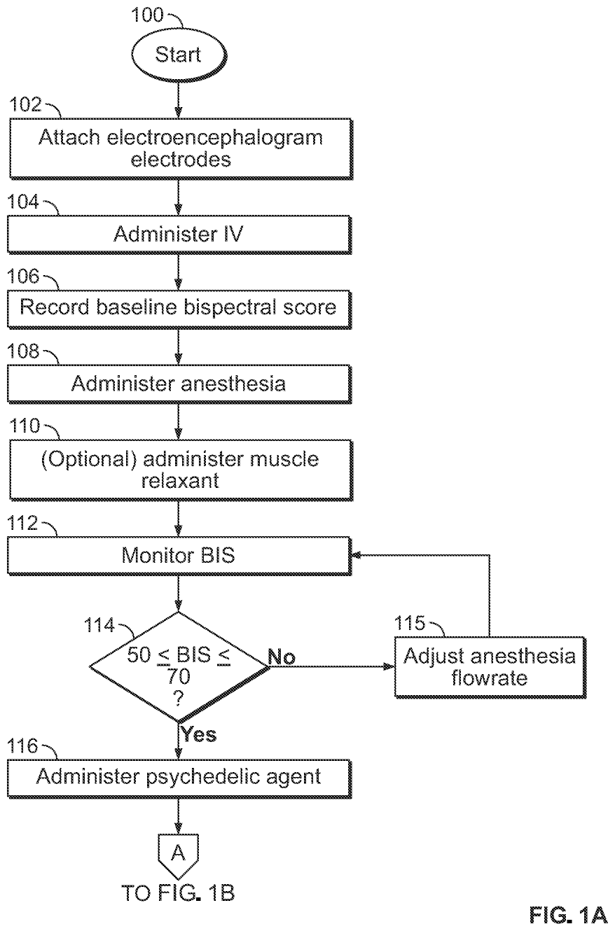

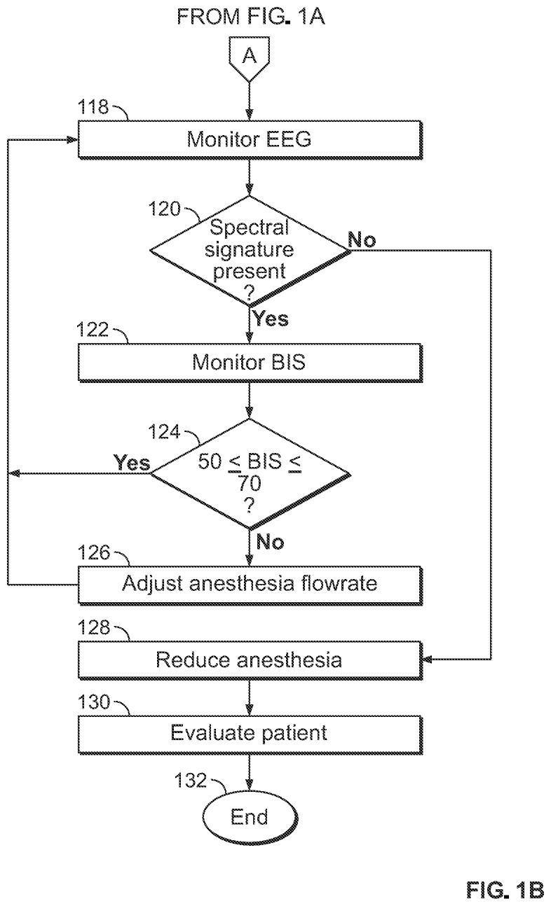

Referring to FIGS. 1A and 1B, the preferred embodiment of the method for treating mental health disorders will be further described. Unless otherwise specified, all tolerances, and usages of the term “about” refer to ±10%.

At step 100, the method begins.

At step 102, electroencephalogram electrodes are applied and, if necessary, affixed to the forehead using collodion adhesive to monitor brain activity. The electrodes convert time domain EEG activity into a frequency domain via a fast Fourier or a wavelet analysis which is displayed on a smart device or computer.

At step 104, an IV is initiated and administered. Preferably a 20 g IV is administered using D5½NS running about 125 ml/hr.

At step 106, EEG data is observed and recorded as a baseline biomarker bispectral index score (BIS).

At step 108, anesthesia is administered to create a state of unconsciousness that eliminates or minimizes conscious awareness or recall of the hallucinogenic experience. In the preferred embodiment, the anesthetic is Diprivan, also known as Propofol, is administered at a dosage of about 0.45 mcg/ml for induction and maintained at a dosage of about 0.9 mcg/ml. In another embodiment, similar short duration, fast acting anesthetics that minimally impact the function of serotonin receptors may be used, such as Sevorflurane, also known as flouromethyl.

At step 110, a muscle relaxant is optionally administered.

At step 112, the BIS is monitored. Preferably a BIS of between about 50 and about 70 is desired. Ideally, a BIS of about 60 is maintained. A BIS of between about 50 and about 70 represents the depth of anesthesia needed to produce the amnestic state that prevents memory formation and recall. A BIS of about 100 indicates an awake state.

At step 114, if the BIS range is between about 50 and about 70, then the method proceeds to step 116. If not, the method proceeds to step 115.

At step 115, the flowrate of the anesthesia is adjusted to bring the BIS into the desired range. The method then returns to step 112.

At step 116, the psychedelic drug is administered. Preferably, 5-MeO-DMT is administered intravenously. Preferably, the effective dose ranges between about 0.5 mg and about 2 mg. The administration of 5-MeO-DMT creates a short and intense hallucinogenic experience. In another embodiment, the psychedelic drug may be administered intramuscularly with a preferred effective dosage range between about 1.4 mg and about 10 mg to produce a hallucinogenic experience.

At step 118, EEG data is monitored to determine the presence of a hallucinogenic state in the patient. Typically, a hallucinogenic state is characterized by a spectral signature with a 8 Hz theta band peak in the EEG spectrum and a large increase of EEG coherence in both the delta band (1-4 Hz) and high beta/low gamma band (25-30 Hz). The hallucinogenic state typically lasts between about 10 to about 20 minutes.

At step 120, if the desired spectral signature is present, then the method proceeds to step 122. If not, then the method proceeds to step 128.

At step 122, if the desired spectral signature is present, the BIS is again monitored.

At step 124, if the BIS range is between about 50 and about 70, then the method returns to step 118. If not, then the method proceeds to step 126.

At step 126, the flowrate of the anesthesia is adjusted to bring the BIS into the desired range. The method then returns to step 118.

At step 128, the anesthesia is reduced until a BIS of between about 90 and about 100 is achieved, and the patient is returned to an awake state.

At step 130, the patient is evaluated for procedure efficacy.

At step 132, the method concludes.

The present invention can be executed by other than the stated preferred embodiments. The preferred embodiments are included for illustration purposes only and do not define a singular set of parameters. Other similar parameters or stimulus presentation paradigms can be included as part of this invention that will provide similar outcomes or augment existing outcomes as described above. It should be understood similar parameter values can be used without substantively deviating from the intent or scope of the invention.

1. A method for treating a patient with mental health disorders, comprising:
providing an EEG device for monitoring and displaying brain activity data of the patient;
administering an IV to the patient;
identifying a bispectral index score (BIS) from the EEG device;
administering an effective dose of an anesthesia to bring the BIS into a BIS range of between about 50 and about 70;
administering an effective dose of a psychedelic drug for producing a hallucinogenic state until a predetermined spectral signature is present;
maintaining the BIS range until the predetermined spectral signature is not present; and
reducing the effective dose of the anesthesia until the BIS is at least about 90.

2. The method of claim 1, wherein the step of administering the effective dose of the anesthesia further comprises:
providing a 20 g IV using D5½NS running at about 125 ml/hr.

3. The method of claim 1, wherein the step of administering the effective dose of the anesthesia further comprises:
providing the anesthesia having no effect on 5-HT1a serotonin receptors, 5-HT2a serotonin receptors, and Sigma-receptors.

4. The method of claim 3, wherein the step of administering the effective dose of the anesthesia further comprises:
administering one of a group of Propofol, Isoflurane, and Sevoflurane.

5. The method of claim 1, wherein the step of administering the effective dose of the psychedelic drug further comprises:
providing the psychedelic drug as having a set of properties which alter activity at serotonin receptors and increase neurogenesis and synaptic plasticity.

6. The method of claim 5, wherein the step of administering the effective dose of the psychedelic drug further comprises:
administering one of a group of 5-MeO-DMT, 5-MeO-DALT, 6-MeO-isoDMT and taberanthalog.

7. The method of claim 1, wherein the step of administering the effective dose of the psychedelic drug further comprises:
administering the psychedelic drug in one of a group of intravenously and intramuscularly.

8. The method of claim 1, wherein the step of administering the effective dose of the anesthesia further comprises:
administering a muscle relaxant agent.

9. A method for treating a patient with mental health disorders, comprising:
providing an EEG device for monitoring and displaying brain activity data of the patient;
administering a 20 g IV using D5½NS running at about 125 ml/hr to the patient;
identifying a bispectral index score (BIS) from the EEG device;
administering an effective dose of an anesthesia to bring the BIS into a BIS range of between about 50 and about 70;
administering a muscle relaxant agent;
administering an effective dose of a psychedelic drug for producing a hallucinogenic state until a predetermined spectral signature is present;
maintaining the BIS range until the predetermined spectral signature is not present; and
reducing the effective dose of the anesthesia until the BIS is at least about 90.

10. The method of claim 9, wherein the step of administering the effective dose of the anesthesia further comprises:
providing the anesthesia as Propofol.

11. The method of claim 10, wherein the step of administering the effective dose of the anesthesia further comprises:
administering Propofol at a dosage of about 0.45 mcg/ml at induction and about 0.9 mcg/ml for maintenance.

12. The method of claim 9, wherein the step of administering the effective dose of the psychedelic drug further comprises:
providing the psychedelic drug as 5-MeO-DMT.

13. The method of claim 12, wherein the step of administering the effective dose of the psychedelic drug further comprises:
administering the 5-MeO DMT intravenously at a dosage between about 0.5 mg and about 2 mg.

14. The method of claim 12, wherein the step of administering the effective dose of the psychedelic drug further comprises:
administering the 5-MeO-DMT intramuscularly at a dosage between about 1.4 mg and about 10 mg.

15. A method for treating a patient with mental health disorders, comprising:
providing an ambulatory EEG device for monitoring and displaying brain activity data of the patient;
administering a 20 g IV using D5½NS running at about 125 ml/hr to the patient;
identifying a bispectral index score (BIS) from the ambulatory EEG device;
administering an effective dose of Propofol via an infusion pump at about 0.45 mcg/ml at induction and about 0.9 mcg/ml for maintenance to bring the BIS into a BIS range of between about 50 and about 70;
administering a muscle relaxant agent;
administering an effective dose of a psychedelic drug 5 MeO-DMT intravenously between about 0.5 and about 2 mg for producing a hallucinogenic state until a predetermined spectral signature is present;
maintaining the BIS range until the predetermined spectral signature is not present; and
reducing the effective dose of Propofol until the BIS is at least about 90.

16. The method of claim 15, wherein the step of administering the effective dose of the psychedelic drug 5 MeO-DMT further comprises:
monitoring an EEG spectrum.

17. The method of claim 16, wherein the step of administering the effective dose of the psychedelic drug 5 MeO-DMT further comprises:
providing the hallucinogenic state has an EEG spectral signature characterized about an 8 Hz theta band peak in the EEG spectrum and an increase in EEG coherence between a delta band and high beta/low gamma band.

18. The method of claim 15, wherein the step of administering the effective dose of the psychedelic drug 5 MeO-DMT further comprises:
providing the hallucinogenic state persists for about 10 to about 20 minutes.

19. The method of claim 15, wherein the step of administering the effective dose of Propofol further comprises:
producing an unconscious state characterized by a suppression of neural activity above about 20 Hz with brain activity concentrated in a high alpha, low beta frequency range between about 12 Hz and about 15 Hz, and an elevated low frequency band between about 0.1 Hz and about 2 Hz.

20. The method of claim 17, wherein the step of administering the effective dose of the psychedelic drug 5 MeO-DMT further comprises:
administering the effective dose of psychedelic drug 5-MEO-DMT intramuscularly at an effective dosage of between about 1.4 mg and about 10 mg.
